(19) United States
(12) Patent Application Publication
(10) Pub. No.: US 2019/0008685 A1
(43) Pub. Date: Jan. 10, 2019

(54) EAR CLEANER

(71) Applicant: MOHAMMAD AL-BAKKOUR, WEST BLOOMFIELD, MI (US)

(72) Inventor: MOHAMMAD AL-BAKKOUR, WEST BLOOMFIELD, MI (US)

(21) Appl. No.: 15/945,716

(22) Filed: Apr. 4, 2018

Related U.S. Application Data
(63) Continuation of application No. 14/866,129, filed on Sep. 25, 2015, now abandoned.

(51) Int. Cl. A61F 11/00

(52) U.S. Cl. CPC A61F 11/006

(57) ABSTRACT

The present invention is an ear cleaner designed to have limited insertion into the ear canal so as not to impact ear wax, also known as cerumen, or damage the ear drum. The ear cleaner has several possible form-factors. All form-factors are designed to be fit over the user's finger. The user's finger is the limiting factor in how far the ear cleaner is inserted. The ear cleaner has a limited surface area. The ear cleaner can have one or more features to entrap ear wax. The ear cleaner is made from a coarse woven fabric to entrap ear wax. The ear cleaner is impregnated with an antimicrobial agent, a lubricant, and a stiffener.

### CLAIM OF PRIORITY

This non-provisional U.S. utility patent application claims priority to non-provisional U.S. utility patent application Ser. No. 14/866,129, as a continuation.

### FIELD OF INVENTION

This invention relates to the classes and sub-classes Surgery and Means for cleaning eye, ear or nose. Specifically, this invention relates to the classes and sub-classes for devices to remove cerumen from the ear canal.

### BACKGROUND

Ear wax, also called cerumen, is an organic material produced by the body to protect and lubricate the ear canal. Technically, cerumen is a secretion of both the ceruminous and sebaceous glands, containing fatty acids, desquamated keratinocytes and keratin. In lay terms, a secretion of fatty acids and other organic oils mixes with shed layers of dead skin and hair in the ear canal, itself. Cerumen, a waxy material composed mostly of keratin, coats the ear canal, and provides antibacterial protection as well as lubrication. This wax is supposed to trap dust and dirt particles to keep them from reaching the eardrum, the membrane that separates the ear canal and the middle ear.

The body is continuously producing cerumen. Most of the time, the old ear wax is moved through the ear canal by motions from chewing and other jaw movements. Alternately, cerumen will sluff off the ear canal as a natural result of layers of skin sluffing off, as the skin of the ear canal grows from the inside out. In either event, it ultimately reaches the outside of the ear and flakes off. Ear wax is produced in the outer part of the ear canal, not deep inside the ear.

Since ear wax is produced only in the outer portion of the ear canal, it should normally not be found near the ear drum. The outer portion of the ear canal is conical, or funnel-shaped, while the inner portion of the ear canal is a curved tube terminating in the ear drum. When a person has wax blockage against the ear drum, it is most likely caused by the person probing the ear canal with cotton swabs, twisted napkins or tissue, hair pins, or other instruments which can fit inside the canal. All of these instruments fail in effectively removing the majority of ear wax, and result only in the ear wax being pushed further into the ear canal. Over time, the cerumen accumulates from repeated efforts at cleaning. This results in impacted wax, or a wax blockage, against the ear drum.

Impacted wax, or a wax blockage, is one of the most common causes of temporary, reversible, hearing loss. People try to remove impacted wax with a variety of home remedies. The most common is to, again, use a cotton swab. However, cotton swabs only push wax further into the ear canal, exacerbating the situation. Cotton swabs can cause abrasions in the ear canal, which can lead to infection. In extreme cases, people have punctured their ear drum with cotton swabs. Even the maker of the largest brand of cotton swabs, Unilever, directs users, “To clean your ears, stroke Q-tips® cotton swabs gently around the outer ear, without entering the ear canal.” For all of these reasons, the American Academy of Otolaryngology (“AAO”) warns against using cotton swabs to clean ears.

Another method of removing ear wax is ear candles. Despite assurances from their proponents, ear candles are not a safe option of wax removal. Ear candles routinely cause burns when used. Additionally, part of the ear candle can break off in the ear canal, creating a more intractable obstruction. Ear candles also can penetrate the ear drum. For all of these reasons, the American Academy of Otolaryngology (“AAO”) warns against using ear candling to clean ears. Additionally, the U.S. Food and Drug Administration (“FDA”) issued an advisory in 2010 noting that ear candling carries a significant risk of injury. The FDA notes that there are currently no controlled studies or other scientific evidence that support the safety and effectiveness of ear candles for any of the purported claims or intended uses as contained in the labeling.

According to the AAO, the only safe way to clean the ears is to wash the external ear with a cloth, without inserting anything into the ear canal. If the ear canal becomes blocked with cerumen, the AAO recommends, first, using a home treatment to soften the wax, such as placing a few drops of mineral oil, baby oil, glycerin, or commercial ear drops into the ear canal. Detergents drops, such as hydrogen peroxide or carbamide peroxide, may also be used to soften and remove ear wax. If the ear wax is intractable, irrigation is needed. The AAO recommends using a syringe or commercially available irrigation product, either at home or in the doctor's office. If a syringe is used, saline, de-ionized water, or distilled water should be used. Water or saline should be warmed to body temperature to minimize any side effects, such as dizziness.

With clear direction from the AAO, and most family doctors, there are still many people using cotton swabs to clean their ears. The ear canal has a large number of nerve endings, meaning that cleaning the ear is pleasurable for many people. Additionally, for people who have routinely cleaned their ears, the habit is hard to stop. When most of the wax is removed from the ear canal, the ear canal will itch more and sensation within the ear canal will be more noticeable. As a result, ear canal cleaning is self-reinforcing behavior. Once you clean your ear canal, you start to like the sensation, and you need to repeat the activity because the ear canal will often itch. With so many people insistent on cleaning their ear canal, the market needs a method that satisfies the desire to clean the ear, without causing wax impaction or ear drum damage. Unfortunately, the current state of the art does not have any such device.

There are many different types of ear cleaners in the prior art. Criticisms of cotton swabs have led to a substantial amount of prior art patents that attempt to improve on the traditional cotton swab. U.S. Utility Pat. No. 6,432,117 by named inventor Murray, issued Aug. 13, 2002, is entitled, “Ear cleaning device” (“Murray '117”). Murray '117 teaches a device that has a helical, extensible shaft fitted with a bulbous stopper. The extensible shaft will only insert a compressed, molded and formed piece of cotton a fixed length passed the bulbous stopper, limiting the distance that the cotton can be inserted into the ear canal. Murray '117 is unduly complex, bulky, and costly, and has no market presence with the consumer. U.S. Utility Pat. No. 5,807,301 by named inventor Nadam, issued Sep. 15, 1998, is entitled, “Disposable device for safe cleaning of the ear” (“Nadam '301”). Nadam '301 teaches a thimble-like device with a short shaft protrusion terminating with a cotton swab. Nadam '301 is a dangerous device, because the shaft and cotton swab can be easily jabbed through the ear drum. Placing a swab at the end of a thimble would tend to make it easier, not harder, to perforate the ear drum, due to the relative leverage capable of being applied by an index finger. Additionally, Nadam '301 would tend to have the same limitations of a cotton swab in terms of impacting ear wax. U.S. Utility Pat. No. 7,500,981 by named inventor Jubrail, issued Mar. 10, 2009, is entitled, “Ear cleaning device” (“Jubrail '981”). Jubrail '981 teaches a device that is essentially an electronic cotton swab. A shaft, with a cotton swab on one end, is inserted into an aperture in an electronic base. Jubrail '981 discloses a vibrating base, in which the cotton swab is agitated about the ear canal. Jubrail '981 causes wax to be impacted, just like a standard cotton swab. Additionally, due to the vibration and the remote tactile feedback on the hand, Jubrail '981 is more likely to puncture the ear drum than a traditional cotton swab.

There are several prior art references that attempt to overcome the drawbacks of cotton swabs by putting something novel on the end of a shaft. U.S. Utility Pat. No. 7,347,865 by named inventor DiCecco, issued March 25, 2008, is entitled, “Ear duct cleaning device” (“DiCecco '865”). DiCecco '865 teaches a device for removing impacted wax. DiCecco '865 shows an inflatable bladder on the end of a shaft. The device is inserted into the ear duct, puncturing the impacted wax. The bladder is then inflated so that it will pull the impacted wax out. DiCecco '865 has several drawbacks. First, it assumes that the impacted wax is in the duct, with room between the impacted wax and the ear drum. That is not always the case. Second, DiCecco '865 assumes that it can pierce an impacted blockage of wax without further pushing it into the canal. DiCecco '865 will cause more harm than good in many, if not most, circumstances. Additionally, it does not really appear to be practical for repeated consumer use. U.S. Utility Pat. No. 5,374,276 by named inventor Lay, issued Dec. 20, 1994, is entitled, “Ear wax remover” (“Lay '276”). Lay '276 teaches an ear wax removing device that has a shaft. On one end of the shaft there is a mushroom-shaped extractor head composed of three concentric discs with frusto-conical edges. In theory, the extractor is made of material that will easily deform as it is inserted into the ear canal, and the frusto-conical edges will collect wax as the device is extracted. Lay '276 can still be jammed through the ear drum, because the only rigid portion of the device is the shaft. Additionally, Lay '276 cannot be cheaply made. Lastly, the frusto-conical edges are likely to more abrasive to the ear canal as the device is extracted, than standard cotton swabs.

U.S. Utility Pat. No. 5,888,199 by named inventors Karell, et. al., issued Mar. 30, 1999, is entitled, “Ear cleaning device with a flexion part” (“Karell '199”). Karell '199 teaches a device that has a shaft with a loop on the end, rather than a cotton applicator. The loop is intended to collect wax, to keep it from getting impacted. Karell '199 has a couple of problems. First, it can still be inserted into and through the ear drum. Second, an open loop may agitate the ear wax, but it is of suspect use in removing a large amount of wax. Third, unless great care it taken when forming the loop, Karell '199 will be much more likely to cause abrasions of the ear canal than a traditional cotton swab. U.S. Utility Pat. No. 6,695,802 by named inventor Thompson, issued Feb. 24, 2004, is entitled, “Ear cleaner device” (“Thompson '802”). Thompson '802 teaches a device which has a shaft, similar to a cotton swab. On the end of the shaft is a small open cup, to collect cerumen. U.S. Utility Pat. No. 7,074,230 by named inventor Olson, issued Jul. 11, 2006, is entitled, “Ear cleaning device” (“Olson '230”). Olson '230 is similar to Thompson '802. Olson '230 teaches a shaft with a scoop on one end. In one embodiment, the other end of the shaft has a cotton swab. Thompson '802 and Olson '230 both suffer from the same problems as Karell '199: they can be inserted through the ear drum; they may agitate the wax, but due to the size of the cup or scoop, they won't remove much wax; and the cup or scoop is more likely to cause ear canal abrasions than a typical cotton swab.

There are several design patents, which make no functional claims, that are variations on the cotton swab. U.S. Design Patent Number D490,523 by named inventor Samborski, issued May 25, 2004, entitled, “Ear cleaning tool,” teaches an ornamental design that has a shaft with a loop on either end. U.S. Design Patent Number D489,133 by named inventor Shimizu, issued Apr. 27, 2004, entitled, “Ear cleaner,” teaches an ornamental design for a helical protrusion on the end of a shaft. U.S. Design Patent Number D489,131 by named inventor Gojcaj, issued Apr. 27, 2004, entitled, “Ear cleaner,” teaches an ornamental design for a plurality of flagellates at the end of a shaft. U.S. Design Patent Number D489,131 by named inventor Shimizu, issued Oct. 17, 2000, entitled, “Ear cleaner,” teaches an additional ornamental design for an alternate helical protrusion on the end of a shaft. U.S. Design Patent Number D296,005 by named inventor Alkire, issued May 31, 1988, entitled, “Ear cleaner,” teaches a cotton swab with a curved end. U.S. Design Patent Number D293,378 by named inventor Alkire, issued Dec.22, 1987, entitled, “Ear cleaner,” teaches a cotton swab with a curved end and a bulb-shaped tip.

None of the aforementioned prior art references provides a suitable market solution. The market needs an inexpensive ear cleaner, that removes ear wax. The ear cleaner should be able to be placed in the ear canal, but not reach the ear-drum. The ear cleaner should not abrade the ear canal during cleaning. Currently, there are no prior art references that achieves these relatively simple design criteria.

### SUMMARY

The present invention fills a hole in the current prior art. The present invention is an ear cleaner that fits over the index finger, thus limiting how far it can be inserted into the ear canal. The ear cleaner is made from a piece of coarse woven fabric. The coarse woven fabric is single layer of woven cotton or blended polyester cotton material, such as coarse cheesecloth or gauze. The National Textile Association (“NTA”) of the United States defines the two dimension of a piece of cloth as warp and weft. A piece of cloth has a thread count, given by the number of threads in an inch of the warp and the number of threads in an inch of the weft. For example, coarse cheesecloth would have a thread count of between 20×12 and 28×24. Gauze is a material that has a pair of weft threads that are crossed before and after each warp thread. Coarse gauze can have a thread count of between 10×10 and 20×20.

The ear cleaner is impregnated with a topical anti-microbial agent such as hydrogen peroxide, carbamide peroxide, tetracycline, bacitracin, neomycin, and polymixin B. The ear cleaner can also be impregnated with a lubricating agent such as glycerin, mineral oil, or baby oil. The ear cleaner can also be impregnated with a stiffening agent, such as a phenolic acid, a starch, or a polymer. Such stiffening agents being well-known in the textile industry.

The ear cleaner is intended to be inserted into an ear canal on the tip of the user's finger. When inserted into an ear canal, the coarse woven fabric impregnated with a stiffener abrades the ear canal, dislodging cerumen. The dislodged cerumen is captured in the coarse woven fabric due to the weave of the fabric, itself, and due to the lubricating agent. When the ear cleaner is removed from the ear, the dislodged cerumen is also removed from the ear canal. This distinguishes the present invention from the prior art. The present invention captures and removes the cerumen without resorting to flooding the ear canal. The present invention does not push the cerumen further into the ear canal, compacting the cerumen against the ear drum.

The ear cleaner can be implemented in any form factor. Two such form factors are presented here, to teach and disclose the invention. In the first form factor, the ear cleaner is a circular planar piece of flexible coarse woven fabric made from a single layer. This form factor of the present invention has a surface area of no more than 200 cm². This corresponds to a radius of no more than three-and-one-eighth inches (3⅛″). The ear cleaner is sized so that it can be draped over the user's finger. The coarse woven fabric impregnated with a stiffener, is stiff but pliable. A user can insert the ear cleaner into the outer portion of the ear canal, and the stiffener and coarse woven fabric force the present invention against the user's ear canal. The present invention dislodges cerumen in this way. The coarse woven fabric accumulates cerumen.

In the other form factor, the ear cleaner is a frusto-conical. One end of the frusto-conical surface is connected to, and continuous with, a substantially planar, substantially circular, top surface. The other end of the frusto-conical lateral surface is open. The open end has a flange in order to create hoop stress and give the ear cleaner some rigidity. The ear cleaner is sized so that the open end can be placed over a user's finger. The height of the ear cleaner, from the flange to the top surface, is 0.75″. The user inserts the frusto-conical ear cleaner into the outer portion of the ear canal. The coarse woven fabric, stiffener, and hoop stress force the present invention against the walls of the user's ear canal, dislodging cerument. The coarse woven fabric accumulates the cerumen.

### BRIEF DESCRIPTION OF THE DRAWINGS

The present invention, an ear cleaner, is illustrated with six figures on four sheets. FIG. 1A is a perspective view of the present invention using a frusto-conical form factor. FIG. 1B is a lateral in situ side drawing of the present invention on a user's finger.

FIG. 2 is a top view of the present invention.

FIG. 3 is a bottom view of a first embodiment of the present invention.

FIG. 4 is a top view of the present invention using a circular planar form factor.

FIG. 5 is a perspective view of the circular planar form factor of the present invention being flexed.

FIG. 6 is a bottom view of the present invention.

### DETAILED DESCRIPTION

The following descriptions are not meant to limit the invention, but rather to add to the summary of invention, and illustrate the ear cleaner. The ear cleaner comes in four embodiments, each of which will be discussed.

FIG. 1A shows the present invention, an ear cleaner 100 with a frusto-conical form factor. This form-factor of the ear cleaner 100 is thimble-like, with a top surface 3, a lateral surface 9, a lateral edge 5, a rim 8, a flange 6, and a flange perimeter 7. A frusto-conical shape is a cone with the top removed. The top surface 3 has a dimple 1. The periphery 2 of the dimple is coplanar with the remainder of the top surface 3. The rest of the dimple 1 is indented, and therefore not coplanar with the top surface 3.

The ear cleaner 100 is made from a single layer of coarse woven cotton or polyester and cotton blend fabric. The exterior surface of the ear cleaner 100, specifically the lateral surface 9 and top surface 3 are made from coarse, open-weave fabric. A coarse, open-weave fabric is more likely to entrap cerumen. A stiffener, such as phenol, is used to help the fabric hold its shape and to assist in dislodging cerumen from the ear canal. The ear cleaner 100 is impregnated with common over-the-counter antimicrobial agents such as hydrogen peroxide, carbamide peroxide, tetracycline, bacitracin, neomycin, or polymixin B. Additionally, the ear cleaner 100 is impregnated with a lubricating agent such as glycerin, mineral oil, or baby oil.

FIG. 1B shows the present embodiment of the ear cleaner 100 used in situ on the end of a user's finger 200. The lateral surface 9 and flange perimeter 7 are visible in this view. When inserted into an ear canal (not shown), the size of the finger limits the penetration of the ear cleaner 100 into the ear canal (not shown). The height 88 of this form-factor is, at most, 0.75″. Being on the end of the user's finger 200, the ear cleaner 100 is assured of stopping well short of the ear drum. Additionally, the combination of a coarse woven fabric, a stiffener, and a lubricating agent dislodges and traps cerumen.

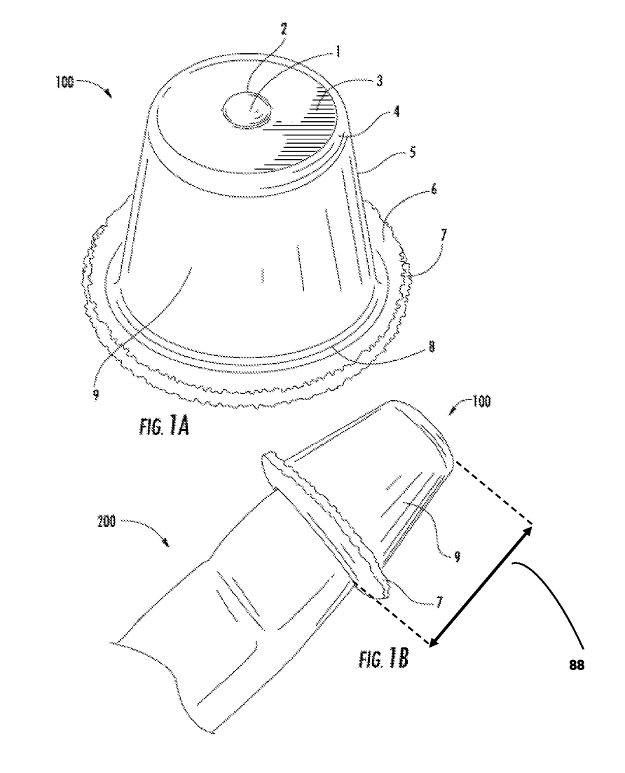

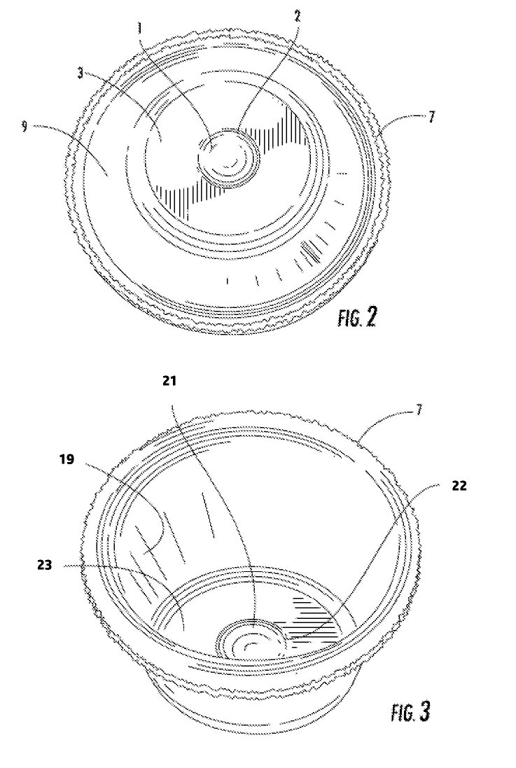

FIG. 2 shows a top view of the frusto-conical form-factor of the ear cleaner 100. The top surface 3, dimple 1, the periphery 2 of the dimple 1, the lateral surface 9 and the flange perimeter 7 are all visible. FIG. 3 shows a bottom view of the first embodiment of the ear cleaner 100. The interior of the lateral surface 19, the interior of the top surface 23, the interior surface of the dimple 21, the interior periphery 22 of the dimple 21, and the flange perimeter 7 are all visible. Since the coarse woven fabric is a single layer, the interior surface 19 is the same material, but the obverse surface, as the exterior surface 9.

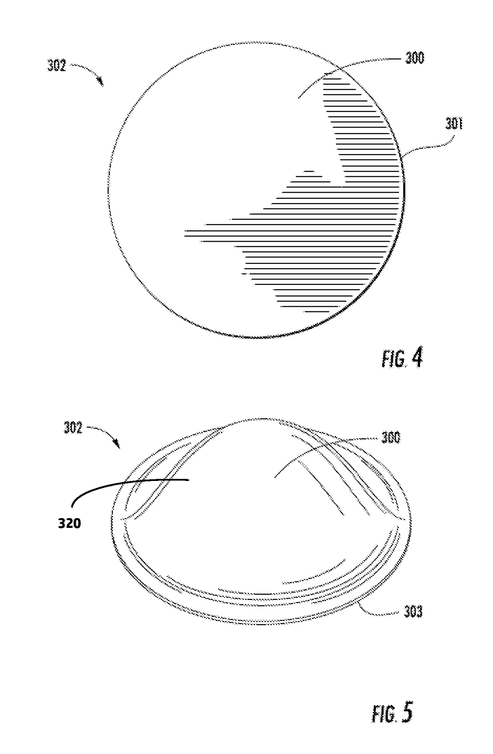

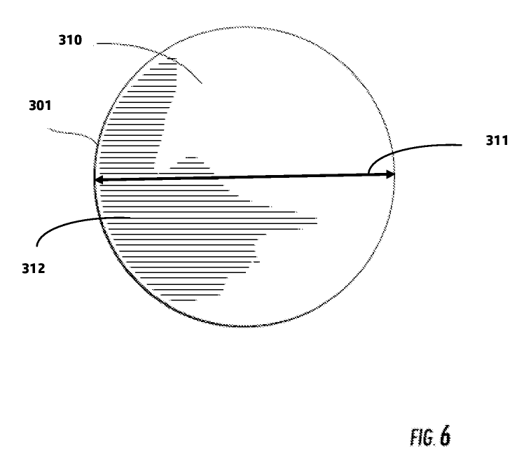

FIG. 4 shows a top view of a circular planar form-factor of the ear cleaner 302. The ear cleaner has a surface 300 and a perimeter 301. FIG. 5 shows a perspective view of the circular planar form-factor of the ear cleaner 302, showing that it would drape 320 around a finger. The top surface 300 and thickness 303 can be seen. FIG. 6 is a bottom view of a circular planar form-factor of the ear cleaner 302. The bottom surface 310 is visible as is the perimeter 301. The ear cleaner 302 has a diameter 311. The maximum diameter 311 of the ear cleaner 302 is approximately 3″. This means the maximum surface 310 area is approximately 9 in² or 60 cm².

The ear cleaner 302 is made from a single layer of coarse woven cotton or polyester and cotton blended fabric. A stiffener, lubicating agent, and anti-microbial are impregnated into the coarse woven fabric of the ear cleaner 302. Common stiffeners suitable for the present invention are phenolic acid (phenol), starch, or polymers. Common microbial agents suitable for the present invention are hydrogen peroxide, carbamide peroxide, tetracycline, bacitracin, neomycin, or polymixin B. Common lubricating agents suitable for the present invention are glycerin, mineral oil, or baby oil.

I claim:

1. An ear cleaner comprised of a single layer of coarse woven fabric, impregnated with a topical anti-microbial, a lubricating agent, and a stiffener, wherein, when inserted into an ear canal, the coarse woven fabric and stiffener of the ear cleaner abrade the cerumen, and the coarse woven fabric and lubricating agent trap the cerumen in the coarse woven fabric for removal from the ear canal.

2. The ear cleaner in claim 1, wherein said coarse woven fabric is at least one of cotton and blended polyester.

3. The ear cleaner in claim 2, wherein coarse woven fabric has a thread count of between 10 and 30 threads per inch in the warp direction and between 10 and 30 threads per inch in the weft direction.

4. The ear cleaner in claim 1, wherein the topical anti-microbial is at least one of hydrogen peroxide, carbamide peroxide, tetracycline, bacitracin, neomycin, and polymixin B.

5. The ear cleaner in claim 1, wherein the lubricating agent is at least one of glycerin, mineral, and baby oil.

6. The ear cleaner in claim 1, wherein the stiffener is at least one of a phenolic acid, a starch, and a polymer. The ear cleaner in claim 1, wherein the ear cleaner is a substantially circular planar disk.

8. The ear cleaner in claim 7, wherein the ear cleaner has a diameter of less than 3″.

9. The ear cleaner in claim 1, wherein the ear cleaner is substantially a frusto-conical.

10. The ear cleaner in claim 9, wherein the ear cleaner has a height, from the base of the frusto-conical to the top of the frusto-conical, of no more than 3″.

11. The ear cleaner in claim 7, wherein the lubricating agent is mineral oil and the stiffener is phenolic acid.
